(19) United States
(12) Patent Application Publication
(10) Pub. No.: US 2025/0143993 A1
(43) Pub. Date: May 8, 2025

(54) COSMETIC

(71) Applicant: SHISEIDO COMPANY, LTD., Chuo-ku, Tokyo (JP)

(72) Inventor: Mika YOSHIMURA, Tokyo (JP)

(73) Assignee: SHISEIDO COMPANY, LTD., Chuo-ku, Tokyo (JP)

(21) Appl. No.: 18/719,582

(22) PCT Filed: Jan. 12, 2023
(86) PCT No.: PCT/JP2023/000635

§ 371 Date: Jun. 13, 2024

(30) Foreign Application Priority Data
Jan. 26, 2022 (JP) 2022-010433

(51) Int. Cl. A61K 8/49; A61K 8/44; A61Q 19/00

(52) U.S. Cl. CPC A61K 8/4946; A61K 8/44; A61Q 19/00

(57) ABSTRACT

A subject matter of the present disclosure is to a cosmetic capable of increasing the skin permeability of imidazolidinone derivatives and their salts.

A cosmetic of the present disclosure comprises at least one water-soluble agent selected from the group consisting of an imidazolidinone derivative of the following Formula 1:

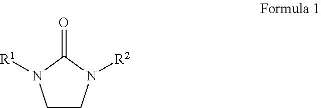

(wherein in Formula 1, R¹ and R² are each independently a hydrogen atom, a C₁ to C₄ linear or branched alkyl group having 0 to 3 hydroxyl groups, or a C₃ to C₇ cycloalkyl group having 0 to 5 hydroxyl groups)
and a salt thereof; and at least one surfactant selected from the group consisting of an amino acid surfactant and a taurine surfactant.

### FIELD

The present disclosure relates to a cosmetic.

### BACKGROUND

In recent years, imidazolidinone derivatives have been used in the fields of cosmetics and the like.

PTL 1 describes a skin cream and a skin care emulsion containing an imidazolidinone derivative.

### CITATION LIST

### Patent Literature

[PTL 1] Japanese Unexamined Patent Publication (Kokai) No. 2008-303186

### SUMMARY

### Technical Problem

The horny layer is positioned in the outermost layer of the skin. Since, for example, it is highly hydrophobic, and has a barrier function that prevents entrance of foreign substances from the external environment, the horny layer is poorly permeable to water-soluble effective components applied to the skin. Therefore, simple application of an agent to the skin tends to let hydrophilic agent components stay on the skin surface, resulting in insufficient production of the effect of the agent.

Among agents, imidazolidinone derivatives and their salts have log Po/w values of about-1.5, and are hence highly hydrophilic. Therefore, the skin does not easily allow permeation of these components, so that a technique that allows permeation of these components into the skin has been demanded.

In view of this, a main object of the present disclosure is to provide a cosmetic capable of increasing the skin permeability of imidazolidinone derivatives and their salts.

### Solution to Problem

### <Aspect 1>

A cosmetic comprising:
at least one water-soluble agent selected from the group consisting of an imidazolidinone derivative of the following Formula 1:

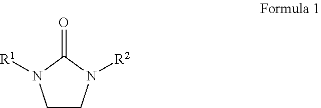

(wherein in Formula 1, R¹ and R² are each independently a hydrogen atom, a C₁ to C₄ linear or branched alkyl group having 0 to 3 hydroxyl groups, or a C₃ to C₇ cycloalkyl group having 0 to 5 hydroxyl groups)
and a salt thereof; and
at least one surfactant selected from the group consisting of an amino acid surfactant and a taurine surfactant.

### <Aspect 2>

The cosmetic according to Aspect 1, wherein the amino acid surfactant is an α-amino acid surfactant.

### <Aspect 3>

The cosmetic according to Aspect 1, wherein the surfactant is at least one selected from the group consisting of lauryl betaine, coconut oil fatty acid amidopropyl betaine, sodium cocoyl glycinate, potassium cocoyl glutamate, and coconut oil fatty acid sodium methyl taurate.

### <Aspect 4>

The cosmetic according to any one of Aspects 1 to 3, wherein the imidazolidinone derivative is at least one selected from the group consisting of 2-imidazolidinone, 1-methyl-2-imidazolidinone, 1,3-dimethyl-2-imidazolidinone, 1-(2-hydroxyethyl)-2-imidazolidinone, and 1,3-bis-(2-hydroxyethyl)-2-imidazolidinone.

### <Aspect 5>

The cosmetic according to any one of Aspects 1 to 4, wherein the mass ratio of the surfactant to the water-soluble agent is more than 0.05.

### <Aspect 6>

The cosmetic according to any one of Aspects 1 to 5, wherein the content of the water-soluble agent in the total cosmetic is 0.1% by mass or more.

### <Aspect 7>

A beauty method comprising applying the cosmetic according to any one of Aspects 1 to 6 to skin.

### <Aspect 8>

Use of at least one surfactant selected from the group consisting of an amino acid surfactant and a taurine surfactant as a transdermal permeation enhancer for at least one water-soluble agent selected from the group consisting of an imidazolidinone derivative of the following Formula 1:

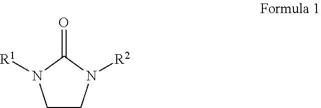

(wherein in Formula 1, R¹ and R² are each independently a hydrogen atom, a C₁ to C₄ linear or branched alkyl group having 0 to 3 hydroxyl groups, or a C₃ to C₇ cycloalkyl group having 0 to 5 hydroxyl groups) and a salt thereof.

### <Aspect 9>

The use according to Aspect 8, wherein the amino acid surfactant is an α-amino acid surfactant.

### <Aspect 10>

The use according to Aspect 8, wherein the surfactant is at least one selected from the group consisting of lauryl betaine, coconut oil fatty acid amidopropyl betaine, sodium cocoyl glycinate, potassium cocoyl glutamate, and coconut oil fatty acid sodium methyl taurate.

### <Aspect 11>

The use according to any one of Aspects 8 to 10, wherein the imidazolidinone derivative is at least one selected from the group consisting of 2-imidazolidinone, 1-methyl-2-imidazolidinone, 1,3-dimethyl-2-imidazolidinone, 1-(2-hydroxyethyl)-2-imidazolidinone, and 1,3-bis-(2-hydroxyethyl)-2-imidazolidinone.

### Advantageous Effects of Invention

According to the present disclosure, a cosmetic capable of increasing the skin permeability of imidazolidinone derivatives and their salts can be provided.

### BRIEF DESCRIPTION OF DRAWINGS

FIG. 1 is a graph showing the ratio of the cumulative amount of permeation in each of examples containing different transdermal-permeation-enhancing components, to the cumulative amount of permeation in Comparative Example 1 or Reference Comparative Example 1, which contains no transdermal-permeation-enhancing component.

FIG. 2 is a graph showing the cumulative amounts of permeation after 3 hours observed in cases where various amino acid surfactants and taurine surfactants were used.

### DESCRIPTION OF EMBODIMENTS

Embodiments of the present disclosure are described below in detail. The present disclosure is not limited to the following embodiments, and may be carried out with various modifications within the scope of the spirit of the present invention.

The cosmetic of the present disclosure comprises: at least one water-soluble agent selected from the group consisting of an imidazolidinone derivative of the above Formula 1 and a salt thereof (which may be hereinafter referred to as “particular water-soluble agent”); and at least one surfactant selected from the group consisting of an amino acid surfactant and a taurine surfactant (which may be hereinafter referred to as “particular surfactant”).

Although the present invention is not limited by any principle, an action principle by which the cosmetic of the present disclosure is capable of increasing the skin permeability (which may be hereinafter referred to as “transdermal permeability”) of the imidazolidinone derivatives and salts thereof is thought to be as follows.

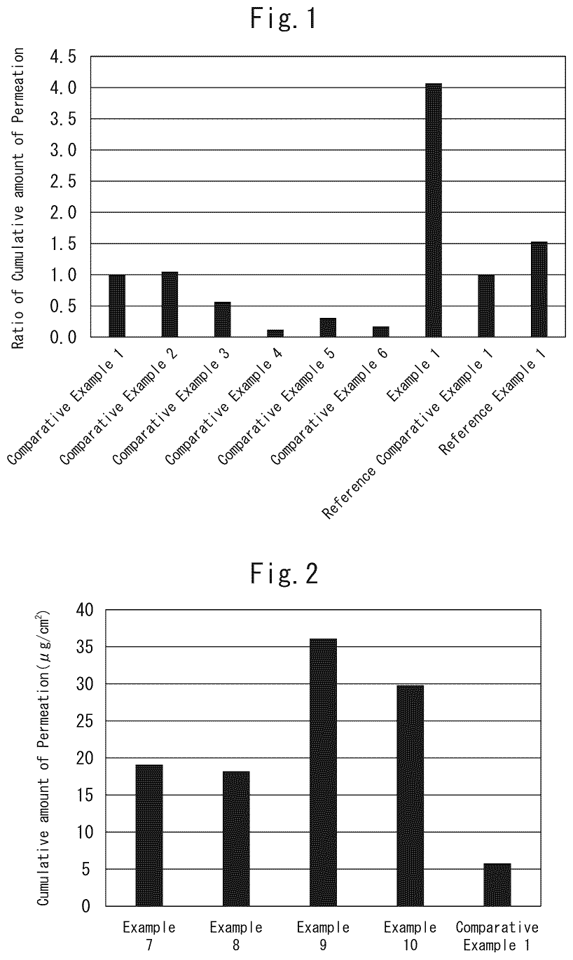

As shown by Comparative Example 1 in FIG. 1 and FIG. 2, the particular water-soluble agent of the present disclosure permeates into a pseudo skin membrane to some extent even without combined use of an agent known as a dermal permeation enhancer. Since the pseudo skin membrane used in the present disclosure has characteristics similar to the properties of the skin, successful permeation of an agent into the pseudo skin membrane may indicate that the agent can similarly permeate into the skin.

On the other hand, as shown by Comparative Examples 4 to 6 in FIG. 1, combined use of an agent that is known to be a dermal permeation enhancer, such as urea or ethanol, with the particular water-soluble agent leads to no improvement in the permeability of the pseudo skin membrane to the particular water-soluble agent, in other words, no improvement in the transdermal permeability.

As can be seen from the results of Reference Comparative Example 1 and Reference Example 1 in FIG. 1, use of, for example, the amino acid surfactant lauryl betaine in combination with niacinamide (nicotinamide), which is a known water-soluble agent, still allows enhancement of the permeability of the pseudo skin membrane, in other words, the transdermal permeability. Surprisingly, however, the present inventors discovered that combined use of lauryl betaine with the particular water-soluble agent allows not less than two-fold improvement in the permeability of the pseudo skin membrane, in other words, the transdermal permeability, compared to the case with niacinamide.

It is thus thought that not every dermal permeation enhancer is effective for enhancement of the skin permeability of the particular water-soluble agent, and that specific enhancement of the skin permeability can be achieved due to a certain interaction only in cases where, among a variety of agents, an amino acid surfactant or a taurine surfactant is used in combination.

### <<Cosmetic>>

The cosmetic of the present disclosure comprises: at least one water-soluble agent (particular water-soluble agent) selected from the group consisting of a imidazolidinone derivative of the above Formula 1 and a salt thereof; and at least one surfactant (particular surfactant) selected from the group consisting of an amino acid surfactant and a taurine surfactant.

### <Particular Water-Soluble Agent>

The particular water-soluble agent in the present disclosure is at least one water-soluble agent selected from the group consisting of an imidazolidinone derivative of the following Formula 1 and a salt thereof. Such an agent is capable of producing, for example, a wrinkle-improving effect, an SCCA1-suppressing effect, or the like.

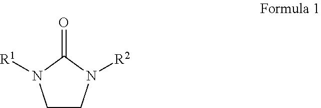

In Formula 1, R¹ and R² are each independently a hydrogen atom, a C₁ to C₄ linear or branched alkyl group having 0 to 3 hydroxyl groups, or a C₃ to C₇ cycloalkyl group having 0 to 5 hydroxyl groups.

In particular, from the viewpoint of further improving the skin permeability in cases of combined use with the particular surfactant, the imidazolidinone derivative(s) is/are more preferably at least one selected from the group consisting of 2-imidazolidinone, 1-methyl-2-imidazolidinone, 1,3-dimethyl-2-imidazolidinone, 1-(2-hydroxyethyl)-2-imidazolidinone, and 1,3-bis-(2-hydroxyethyl)-2-imidazolidinone. 1-(2-Hydroxyethyl)-2-imidazolidinone (which may be referred to as “hydroxyethyl imidazolidinone”) is more preferred.

An imidazolidinone derivative can be converted to an inorganic salt or organic salt by a known method. The salt is not limited. Examples of the inorganic salt include hydrochloric acid salt, sulfuric acid salt, phosphoric acid salt, hydrobromic acid salt, sodium salt, potassium salt, magnesium salt, calcium salt, and ammonium salt. Examples of the organic salt include acetic acid salt, lactic acid salt, maleic acid salt, fumaric acid salt, tartaric acid salt, citric acid salt, methanesulfonic acid salt, p-toluene sulfonic acid salt, triethanolamine salt, diethanolamine salt, and amino acid salt.

The amount of the particular water-soluble agent included is not limited. From the viewpoint of further improving the skin permeability in cases of combined use with the particular surfactant, the amount of the particular water-soluble agent included is preferably 0.1% by mass or more, 0.3% by mass or more, 0.5% by mass or more, 0.7% by mass or more, 1.0% by mass or more, or 1.5% by mass or more with respect to the total amount of the cosmetic. There is no upper limit of the included amount, and the amount may be, for example, 10% by mass or less, 8.0% by mass or less, 5.0% by mass or less, or 3.0% by mass or less.

### <Particular Surfactant>

The particular surfactant in the present disclosure is at least one surfactant selected from the group consisting of an amino acid surfactant and a taurine surfactant. Since the particular surfactant is capable of increasing the skin permeability of the above particular water-soluble agent, the particular surfactant can be used as a transdermal permeation enhancer for the particular water-soluble agent.

The amino acid surfactant in the present disclosure means a surfactant having an amino-acid-derived structure. The amino acid herein is a general term for organic compounds containing an amino group and a carboxy group.

The amino acid surfactant is preferably an α-amino acid surfactant from the viewpoint of further improving the skin permeability in cases of combined use with the particular water-soluble agent.

More preferred specific examples of the amino acid surfactant include lauryl betaine, coconut oil fatty acid amidopropyl betaine, cocoyl glycinates (such as sodium cocoyl glycinate) and cocoyl glutamates (such as potassium cocoyl glutamate), 2-alkyl-N-carboxymethyl-N-hydroxyethyl imidazolinium betaine, cocamidopropyl betaine, lauramidopropyl betaine, cocobetaine, cocoamphoacetates (such as sodium cocoamphoacetate), lauroamphoacetates (such as sodium lauroamphoacetate), stearoyl glutamates, myristoyl glutamates, and lauryl glutamates. These may be used individually, or as a combination of two or more thereof.

The taurine surfactant in the present disclosure means a surfactant having a taurine-derived structure.

Examples of the taurine surfactant include surfactants having a structure derived from acyl taurine or acyl methyl taurine (that is, N-acyl-N-methyl taurine).

Specific examples of the taurine surfactant include at least one selected from the group consisting of caproyl taurine, lauroyl taurine, myristoyl taurine, palmitoyl taurine, stearoyl taurine, oleoyl taurine, cocoyl taurine, methyl taurine, coconut oil fatty acid methyl taurine, palm kernel oil fatty acid methyl taurine, hydrogenated palm kernel oil fatty acid methyl taurine, tallow fatty acid methyl taurine, hydrogenated tallow fatty acid methyl taurine, caproyl methyl taurine, lauroyl methyl taurine, myristoyl methyl taurine, palmitoyl methyl taurine, stearoyl methyl taurine, oleoyl methyl taurine, cocoyl methyl taurine, and methyl taurine cocoyl methyl taurine; and salts thereof. In particular, from the viewpoint of further improving the skin permeability in cases of combined use with the particular water-soluble agent, coconut oil fatty acid methyl taurine or a salt thereof is preferred. Examples of the salt include alkali metal salts such as sodium salt and potassium salt; alkaline earth metal salts such as calcium salt and magnesium salt; zinc salt; iron salt; ammonium salt; salts with a basic amino acid such as arginine, lysine, histidine, or ornithine; and salts with an amine such as monoethanolamine, diethanolamine, or triethanolamine. In particular, alkali metal salts are preferred, and sodium salt is more preferred.

The amount of the particular surfactant included may be, for example, 0.05% by mass or more, more than 0.05% by mass, 0.07% by mass or more, 0.1% by mass or more, or more than 0.1% by mass, and may be, for example, 8.0% by mass or less, 6.0% by mass or less, 5.0% by mass or less, 3.0% by mass or less, 1.0% by mass or less, 0.7% by mass or less, 0.5% by mass or less, or 0.3% by mass or less, with respect to the total amount of the cosmetic.

The particular surfactant is capable of functioning as a transdermal permeation enhancer for the particular water-soluble agent described above. Thus, from the viewpoint of further improving the skin permeability in cases of combined use with the particular water-soluble agent, the mass ratio of the particular surfactant to the particular water-soluble agent is preferably more than 0.05, 0.07 or more, 0.10 or more, or 0.15 or more, and is preferably 5.0 or less, 4.0 or less, 3.0 or less, 2.0 or less, 1.5 or less, 1.0 or less, 0.70 or less, 0.50 or less, or 0.40 or less.

### <Water>

The cosmetic of the present disclosure may comprise water. The water is not limited as long as the particular water-soluble agent can be dissolved therein. For example, water for use in cosmetics may be used. Examples of such water include ion-exchanged water, purified water, distilled water, ultrapure water, and tap water.

The amount of the water included is not limited, and may be appropriately adjusted, for example, in accordance with the formulation (such as the form of a single aqueous phase, or the form of an oil-in-water emulsion or a water-in-oil emulsion) employed for the cosmetic. More specifically, for example, the amount of the water included may be 10% by mass or more, 20% by mass or more, 30% by mass or more, 40% by mass or more, 45% by mass or more, 50% by mass or more, 55% by mass or more, 60% by mass or more, 65% by mass or more, 70% by mass or more, 75% by mass or more, or 80% by mass or more, and may be less than 100% by mass, 99% by mass or less, 98% by mass or less, 95% by mass or less, 90% by mass or less, 80% by mass or less, 70% by mass or less, 60% by mass or less, or 50% by mass or less, with respect to the total amount of the cosmetic.

### <Optional Components>

The cosmetic of the present disclosure may contain various components as appropriate, as long as the components do not adversely affect the effect of the present disclosure. Examples of the components include surfactants (such as anionic surfactants, cationic surfactants, amphoteric surfactants, and nonionic surfactants) other than the particular surfactant described above, thickeners, moisturizers, dispersants, water-soluble polymers, film forming agents, sequestering agents, lower alcohols (such as ethanol), polyols (such as glycerin), higher alcohols, various extracts, sugars, amino acids, organic amines, polymer emulsions, chelating agents, ultraviolet absorbers, pH-adjusting agents, skin nutrients, vitamins, water-soluble agents (such as niacinamide) other than the particular water-soluble agent described above, buffers, preservatives, antioxidants, stabilizers, propellants, fillers, algefacients, pigments, dyes, colorants, perfumes, and oils. The optional components may be used individually, or as a combination of two or more thereof.

In some embodiments, the cosmetic of the present disclosure may contain an alkylene oxide derivative of Formula 2 below. Combined use of such a derivative with the particular surfactant further improves the skin permeability of the particular water-soluble agent.

R^{a}O-[(AO)ₘ(EO)ₙ]—R^{b} Formula 2

In Formula 2, AO is a C₃ to C₄ oxyalkylene group; EO is an oxyethylene group; and m and n are the average numbers of moles of oxyalkylene groups and oxyethylene groups added, respectively, satisfying 1≤m≤70 and 1≤n≤70. The ratio of the oxyethylene groups to the total of the C₃ to C₄ oxyalkylene groups and the oxyethylene groups is 20 to 80% by mass. The C₃ to C₄ oxyalkylene groups and the oxyethylene groups may be added in blocks or in a random manner. R^{a} and R^{b} may be the same or different, and are each a C₁ to C₄ hydrocarbon group or a hydrogen atom, wherein the ratio of the number of the hydrogen atoms to the number of the hydrocarbon groups of R^{a} and R^{b} is 0.15 or less.

Specific examples of AO include oxypropylene, oxybutylene, oxyisobutylene, oxytrimethylene, and oxytetramethylene. In particular, oxypropylene and oxybutylene are preferred.

m, which is the average number of moles of C₃ to C₄ oxyalkylene groups added, is preferably within the range of 2≤m≤20. n, which is the average number of moles of oxyethylene groups added, is preferably within the range of 2≤n≤20. From the viewpoint of stickiness and the like, (m+n) is preferably within the range of 8 to 100.

In Formula 2, the order of the EO's and AO's added is not limited, and these may be added in blocks or in a random manner. In cases where they are added in blocks, the EO's and AO's may be added in two blocks, or may be added in three blocks or more. The EO's and AO's are preferably added in a random manner.

R^{a} and R^{b} are each a C₁ to C₄ hydrocarbon group or a hydrogen atom, and may be the same or different. Examples of the hydrocarbon group include methyl, ethyl, n-propyl, isopropyl, n-butyl, sec-butyl, and tert-butyl. In particular, methyl and ethyl are preferred.

Each of R^{a} and R^{b} may be constituted by a single type of hydrocarbon group, by hydrogen atoms, by a mixture of hydrocarbon groups and hydrogen atoms, or by a mixture of different hydrocarbon groups. However, the ratio of the number of hydrogen atoms (Y) to the number of hydrocarbon groups (X), Y/X, of R^{a} and R^{b} is 0.15 or less, preferably 0.06 or less.

The alkylene oxide derivative of Formula 2 above can be produced by a known method. It can be obtained, for example, by performing addition polymerization of ethylene oxide and C₃ to C₄ alkylene oxide to a compound containing a hydroxyl group, and then performing ether reaction with an alkyl halide in the presence of an alkali catalyst.

The amount of the alkylene oxide derivative of Formula 2 above included is not limited, and may be, for example, 0.1% by mass or more, 0.5% by mass or more, or 1% by mass or more, and may be, for example, 20% by mass or less, 17% by mass or less, 15% by mass or less, 13% by mass or less, 10% by mass or less, 8.0% by mass or less, 6.0% by mass or less, 5.0% by mass or less, or 3.0% by mass or less, with respect to the total amount of the cosmetic.

From the viewpoint of further improving the skin permeability in cases of combined use with the particular water-soluble agent, the mass ratio of the alkylene oxide derivative of Formula 2 above to the particular water-soluble agent is preferably 0.5 or more, 0.7 or more, 1.0 or more, 1.5 or more, or 2.0 or more, and is preferably 20 or less, 15 or less, 12 or less, 10 or less, or 8.0 or less.

### <Skin Permeability of Particular Water-Soluble Agent>

The skin permeability of the particular water-soluble agent in the cosmetic of the present disclosure can be evaluated, for example, from the result of the later-described cumulative permeation test using a pseudo skin membrane. The pseudo skin membrane has characteristics similar to the properties of the skin. Therefore, such a cumulative permeation test enables simulation of the skin permeation performance of the agent.

### (Cumulative Amount of Permeation of Particular Water-Soluble Agent)

In some embodiments, the cosmetic of the present disclosure can achieve a cumulative amount of permeation of 5.0 μg/cm² or more, 7.0 g/cm² or more, 10.0 μg/cm² or more, 13.0 μg/cm² or more, or 15.0 μg/cm² or more after 3 hours in the cumulative permeation test. There is no upper limit of the cumulative amount of permeation, and the cumulative amount of permeation may be, for example, 100 μg/cm² or less, 80.0 μg/cm² or less, 60.0 μg/cm² or less, or 55.0 μg/cm² or less.

### (Ratio Regarding Cumulative Amount of Permeation of Particular Water-Soluble Agent)

In some embodiments, regarding the cumulative amount of permeation after 3 hours in such a cumulative permeation test, the cosmetic of the present disclosure can achieve a ratio of more than 1.5, 1.7 or more, 2.0 or more, 2.3 or more, or 2.5 or more in terms of the ratio of the cumulative amount of permeation of the cosmetic comprising the particular surfactant to the cumulative amount of permeation of a cosmetic not comprising the particular surfactant. There is no upper limit of the ratio, and the ratio may be, for example, 15 or less, 14 or less, 13 or less, 12 or less, 11 or less, 10 or less, 9.0 or less, or 8.0 or less. The “cosmetic not comprising the particular surfactant” in the present disclosure is a cosmetic having substantially the same composition as that of the cosmetic comprising the particular surfactant, except that the particular surfactant is not contained in the former cosmetic. The term “substantially” means that it is acceptable for a surfactant (for example, the nonionic surfactant “PPG-13 decyltetradeceth-24”) having no action as a dermal permeation enhancer, to be included in a predetermined amount (for example, 0.2% by mass) in the entire cosmetic not comprising the particular surfactant. In cases where the cosmetic contains no surfactant, the particular water-soluble agent may be repelled on the surface of the pseudo skin membrane or the skin, so that comparative experiments cannot be appropriately carried out. Thus, in the cosmetic not comprising the particular surfactant, inclusion of a predetermined amount of a surfactant having no action as a dermal permeation enhancer is acceptable.

### <Formulation and Form of Cosmetic>

The formulation of the cosmetic of the present disclosure is not limited. From the viewpoint of the skin permeability of the particular water-soluble agent, the cosmetic is preferably in the form of a single aqueous phase, or in the form of an oil-in-water emulsion or a water-in-oil emulsion. The cosmetic is more preferably in the form of a single aqueous phase. These formulations can be appropriately prepared by a conventional method using a known material such as an oil component, an emulsifier, or water, when necessary. The “single aqueous phase” in the present disclosure means a single phase substantially constituted by an aqueous phase. The term “substantially” means that, for example, slight incorporation of oil components (such as an oil-soluble ultraviolet absorber) into the aqueous phase due to solvation or solubilization by an alcohol or the like is acceptable, but that, for example, oil droplets (emulsion particles) emulsified by a surfactant or the like, such as those contained in an oil-in-water emulsion composition, are not included.

The form of the cosmetic of the present disclosure is not limited, and the cosmetic may be in the form of an emulsion, a cream, or a liquid.

The product form of the cosmetic of the present disclosure is also not limited, and examples of the product form include skin care cosmetics such as beauty essences, skin lotions, moisturizing gels, massage gels, emulsions, and creams; facial cosmetics such as packs; makeup cosmetics such as foundations and eye shadows; sunscreen cosmetics (sunscreen agents); body cosmetics; skin washing products such as makeup removers and body shampoos; hair cosmetics such as hair liquids, hair tonics, hair conditioners, shampoos, hair rinses, and hair restorers; shaving cosmetics such as shaving creams, pre-shaving lotions, and after-shaving lotions; and ointments.

### <<Application Sites for Cosmetic>>

The cosmetic of the present disclosure is applicable to any part of the body. For example, it may be applied to any site on the skin surface (body surface). More specifically, the cosmetic may be applied as appropriate to the skin surface of the face (for example, lip; area around the eyes; eyelids; cheeks; forehead; glabella; or nose), head (scalp), ears, hands, arms, neck, legs, feet, chest, abdomen, or back. In particular, the skin also includes nails and the like formed by hardening of the corneum of the skin epidermis.

### <<Beauty Method Using Cosmetic>>

The beauty method using the cosmetic of the present disclosure includes application of the above-described cosmetic to the skin. In the present disclosure, “beauty method” means a method of applying the cosmetic of the present disclosure to the skin to beautify it by achieving a beautiful and orderly condition of the skin. The method is thus different from a method of operation, treatment, or diagnosis of a human.

The cosmetic for the beauty method of the present disclosure is not limited, and examples of the cosmetic include skin care cosmetics such as beauty essences, skin lotions, moisturizing gels, massage gels, emulsions, and creams. A single cosmetic, or a combination of two or more cosmetics may be used. The cosmetic may be used in a state where a face mask or the like is impregnated therewith.

### EXAMPLES

The present invention is described in more detail by way of Test Examples and Examples. However, the present invention is not limited thereto. Unless otherwise specified, each included amount is expressed in % by mass.

### Test Examples 1 to 4

The evaluation described below was carried out using each sample obtained by the following production method, and the results are summarized in Tables 1 to 4 and FIGS. 1 and 2. For the Comparative Examples and the Examples in each table and FIG. 1, the term “ratio of the cumulative amount of permeation” means the ratio of the cumulative amount of permeation after 3 hours in each case to the cumulative amount of permeation after 3 hours in Comparative Example 1. For Reference Comparative Example 1 and Reference Example 1 in Table 1 and FIG. 1, the above term means the ratio of the cumulative amount of permeation after 3 hours in Reference Example 1 to the cumulative amount of permeation after 3 hours in Reference Comparative Example 1.

### <Evaluation of Cosmetics>

### (Cumulative Permeation Test)

For each agent, evaluation of the permeability was carried out using a diffusion cell array system (manufactured by Ikeda Scientific Co., Ltd.; hereinafter referred to as “diffusion cell”) suitable for the screening, regarding the permeability of a pseudo skin membrane to the particular water-soluble agent.

As the diffusion cell, a diffusion cell array system with an effective permeation area of 0.785 cm² was used. To provide the pseudo skin membrane, that is, the permeable membrane to be placed in the diffusion cell, Strat-M (trademark) membrane (manufactured by Merck Millipore) was cut out using a puncher such that the membrane fits the outer diameter of the donor cell constituting the diffusion cell. The pseudo skin membrane was thoroughly hydrated by immersing it in physiological phosphate buffer (PBS) before its placement in the diffusion cell.

The receiver cell of the diffusion cell was filled with PBS, and the pseudo skin membrane was tightly fixed between the receiver cell and the donor cell using a cell clamp such that entrance of air was prevented. The diffusion cell was placed on a hot plate to keep the surface temperature of the pseudo skin membrane at about 32° C., which corresponds to the skin surface temperature. The membrane was conditioned for about 1 hour while the PBS in the receiver cell was stirred with a stirring bar.

Application of the sample to the pseudo skin membrane was carried out by finite open application assuming the practical use. The sample was uniformly applied to the pseudo skin membrane such that the applied amount was 10 μg/cm². Three hours after the application, PBS was collected from the receiver cell. Using an HPLC system equipped with an LC-MS detector, the cumulative amount of permeation was calculated by quantifying the amount of the particular water-soluble agent in the PBS collected.

### Test Example 1: Effects of Various Components Used with Particular Water-Soluble Agent on Transdermal Permeability

In Test Example 1, effects of various components used with the particular water-soluble agent on the transdermal permeability were studied. The results are summarized in Table 1 and FIG. 1. Each value of the cumulative amount of permeation is the average of the values obtained by performing three times of measurement for each sample. Reference Comparative Example 1 and Reference Example 1 are examples for evaluation of the effect of the particular surfactant on a water-soluble agent other than the particular water-soluble agent, for the purpose of reference.

### Comparative Example 1

According to the blending ratios shown in Table 1, hydroxyethyl imidazolidinone as the particular water-soluble agent and PPG-13 decyltetradeceth-24 as a surfactant were added to ion-exchanged water, and the resulting mixture was stirred with a stirrer, to prepare a composition of a single aqueous phase.

### Comparative Examples 2 to 6, Example 1, Reference Comparative Example 1, and Reference Example 1

The compositions of a single aqueous phase of Comparative Examples 2 to 6, Example 1, Reference Comparative Example 1, and Reference Example 1 were prepared in the same manner as in Comparative Example 1 except that the components and their included amounts were changed as shown in Table 1.

TABLE 1

| | | | | | | | | | Reference | |
|---|---|---|---|---|---|---|---|---|---|---|
| | | Compar- | Compar- | Compar- | Compar- | Compar- | Compar- | | Compar- | |
| | | ative | ative | ative | ative | ative | ative | | ative | |
| | | Exam- | Exam- | Exam- | Exam- | Exam- | Exam- | Exam- | Exam- | Reference |
| Component | Component name | ple 1 | ple 2 | ple 3 | ple 4 | ple 5 | ple 6 | ple 1 | ple 1 | Example 1 |
| Water | Ion-exchanged water | 98.8 | 98.8 | 98.5 | 98.5 | 94.0 | 93.8 | 98.8 | 94.8 | 94.8 |
| Particular | Hydroxyethyl | 1.0 | 1.0 | 1.0 | 1.0 | 1.0 | 1.0 | 1.0 | — | — |
| water-soluble | imidazolidinone | | | | | | | | | |
| agent | | | | | | | | | | |
| Water- | Niacinamide | — | — | — | — | — | — | — | 5.0 | 5.0 |
| soluble agent | | | | | | | | | | |
| Particular | Lauryl betaine | — | — | — | — | — | — | 0.2 | — | 0.2 |
| surfactant | | | | | | | | | | |
| Surfactant | PPG-13 | 0.2 | — | — | — | — | — | — | 0.2 | — |
| | decyltetradeceth-24 | | | | | | | | | |
| Transdermal | Sodium | — | 0.2 | — | — | — | — | — | — | — |
| permeation | dilauramidoglutamide | | | | | | | | | |
| enhancer | lysine | | | | | | | | | |
| | (Dimethylacrylamide/ | — | — | 0.5 | — | — | — | — | — | — |
| | sodium acryloyl | | | | | | | | | |
| | dimethyl taurate) | | | | | | | | | |
| | crosspolymer | | | | | | | | | |
| | Urea | — | — | — | 0.5 | — | — | — | — | — |
| | Ethanol | — | — | — | — | 5.0 | 5.0 | — | — | — |
| | Menthol | — | — | — | — | — | 0.2 | — | — | — |

| | | | | | | | | | |
|---|---|---|---|---|---|---|---|---|---|
| Total | 100 | 100 | 100 | 100 | 100 | 100 | 100 | 100 | 100 |
| Cumulative amount of permeation | 5.8 | 6.1 | 3.3 | 0.7 | 1.8 | 1 | 23.6 | 16.1 | 24.7 |
| after 3 hours (μg/cm²) | | | | | | | | | |
| Ratio of the cumulative amount of | 1.0 | 1.1 | 0.6 | 0.1 | 0.3 | 0.2 | 4.1 | 1.0 | 1.5 |
| permeation | | | | | | | | | |

### (Results)

As is evident from the results in Table 1 and FIG. 1, it could be confirmed that the composition of Example 1, which contains the particular water-soluble agent and the particular surfactant, is capable of achieving not less than two-fold improvement in the permeability of the particular water-soluble agent into the pseudo skin membrane, in other words, the transdermal permeability, compared to the composition of Comparative Example 1, which was prepared using a surfactant having no action as a dermal permeation enhancer.

Further, according to the result on the ratio between the cumulative amounts of permeation in Reference Comparative Example 1 and Reference Example 1, use of the particular surfactant of the present disclosure with a water-soluble agent (niacinamide) that is not included in the particular water-soluble agent of the present disclosure led to only 1.5-fold improvement in the permeability of the water-soluble agent into the pseudo skin membrane. This result also suggests that the combination of the particular water-soluble agent and the particular surfactant specifically improves the permeability into the pseudo skin membrane, in other words, the transdermal permeability.

In Comparative Examples 2 to 6, known components were used as transdermal permeation enhancers. However, these components were found not to improve the permeability into the pseudo skin membrane, in other words, the transdermal permeability. It was thus found that not every component known as a transdermal permeation enhancer is capable of producing a similar effect on the particular water-soluble agent of the present disclosure.

### Test Example 2: Effects of Differences in Blending Ratio of Particular Surfactant on Transdermal Permeability

In Test Example 2, effects of differences in the blending ratio of the particular surfactant on the transdermal permeability were studied. The results are summarized in Table 2. Each value of the cumulative amount of permeation is the average of the values obtained by performing two times of measurement for each sample. Table 2 also shows the results of Example 1 and Comparative Example 1, for the purpose of reference.

### Examples 2 to 4

The compositions of a single aqueous phase of Examples 2 to 4 were prepared in the same manner as in Example 1 except that the amounts of components included were changed as shown in Table 2.

TABLE 2

| | | Comparative | | | | |
|---|---|---|---|---|---|---|
| Component | Component name | Example 1 | Example 2 | Example 1 | Example 3 | Example 4 |
| Water | Ion-exchanged water | 98.8 | 98.9 | 98.8 | 98.5 | 98.0 |
| Particular water-soluble agent | Hydroxyethyl imidazolidinone | 1.0 | 1.0 | 1.0 | 1.0 | 1.0 |
| Particular surfactant | Lauryl betaine | — | 0.1 | 0.2 | 0.5 | 1.0 |
| Surfactant | PPG-13 decyltetradeceth-24 | 0.2 | — | — | — | — |

| | | | | | |
|---|---|---|---|---|---|
| Total | 100 | 100 | 100 | 100 | 100 |
| Mass ratio of the particular surfactant | — | 0.10 | 0.20 | 0.50 | 1.0 |
| to the particular water-soluble agent | | | | | |
| Cumulative amount of permeation | 5.8 | 17.1 | 23.6 | 13.9 | 14.9 |
| after 3 hours (μg/cm²) | | | | | |
| Ratio of the cumulative amount of permeation | 1.0 | 2.9 | 4.1 | 2.4 | 2.6 |

According to the results in Table 2, it was found that the permeability of the particular water-soluble agent into the pseudo skin membrane, in other words, the transdermal permeability, can be remarkably increased even in cases where the amount of the particular surfactant included is decreased or increased relative to that in Example 1.

### Test Example 3: Effects of Differences in Blending Ratio of Particular Water-Soluble Agent on Transdermal Permeability

In Test Example 3, effects of differences in the blending ratio of the particular water-soluble agent on the transdermal permeability were studied. The results are summarized in Table 3. Each value of the cumulative amount of permeation is the average of the values obtained by performing two times of measurement for each sample. Table 3 also shows the results of Example 1 and Comparative Example 1, for the purpose of reference.

### Examples 5 and 6, and Comparative Examples 7 and 8

The compositions of a single aqueous phase of Examples 5 and 6, and Comparative Examples 7 and 8 were prepared in the same manner as in Example 1 or Comparative Example 1 except that the amounts of components included were changed as shown in Table 3.

TABLE 3

| | | Comparative | | Comparative | | Comparative | |
|---|---|---|---|---|---|---|---|
| Component | Component name | Example 7 | Example 5 | Example 1 | Example 1 | Example 8 | Example 6 |
| Water | Ion-exchanged water | 99.7 | 99.7 | 98.8 | 98.8 | 97.8 | 97.8 |
| Particular water-soluble agent | Hydroxyethyl imidazolidinone | 0.1 | 0.1 | 1.0 | 1.0 | 2.0 | 2.0 |
| Particular surfactant | Lauryl betaine | — | 0.2 | — | 0.2 | — | 0.2 |
| Surfactant | PPG-13 decyltetradeceth-24 | 0.2 | — | 0.2 | — | 0.2 | — |

| | | | | | | |
|---|---|---|---|---|---|---|
| Total | 100 | 100 | 100 | 100 | 100 | 100 |
| Mass ratio of the particular | — | 2.0 | — | 0.20 | — | 0.10 |
| surfactant to the particular water- | | | | | | |
| soluble agent | | | | | | |
| Cumulative amount of permeation | 2.4 | 5.5 | 5.8 | 23.6 | 19.3 | 52.2 |
| after 3 hours (μg/cm²) | | | | | | |
| Ratio of the cumulative amount of permeation | 1.0 | 2.3 | 1.0 | 4.1 | 1.0 | 2.7 |

According to the results in Table 3, it was found that the permeability of the particular water-soluble agent into the pseudo skin membrane, in other words, the transdermal permeability, can be remarkably increased even in cases where the amount of the particular water-soluble agent included is decreased or increased relative to that in Example 1.

### Test Example 4: Effects of Differences in Type of Particular Surfactant on Transdermal Permeability

In Test Example 4, effects of differences in the type of the particular surfactant on the transdermal permeability were studied. The results are summarized in Table 4 and FIG. 2. Each value of the cumulative amount of permeation is the average of the values obtained by performing three times of measurement for each sample.

### Examples 7 to 10

The compositions of a single aqueous phase of Examples 7 to 10 were prepared in the same manner as in Example 1 except that the components and their included amounts were changed as shown in Table 4.

TABLE 4

| | | Comparative | | | | |
|---|---|---|---|---|---|---|
| Component | Component name or trade name | Example 1 | Example 7 | Example 8 | Example 9 | Example 10 |
| Water | Ion-exchanged water | 98.8 | 98.8 | 98.8 | 98.8 | 98.8 |
| Particular water-soluble agent | Hydroxyethyl imidazolidinone | 1.0 | 1.0 | 1.0 | 1.0 | 1.0 |
| Particular surfactant | Coconut oil fatty acid amidopropyl betaine | — | 0.2 | — | — | — |
| | Sodium cocoyl glycinate | — | — | 0.2 | — | — |
| | Potassium cocoyl glutamate | — | — | — | 0.2 | — |
| | Coconut oil fatty acid sodium methyl taurate | — | — | — | — | 0.2 |
| Surfactant | PPG-13 decyltetradeceth-24 | 0.2 | — | — | — | — |

| | | | | | |
|---|---|---|---|---|---|
| Total | 100 | 100 | 100 | 100 | 100 |
| Cumulative amount of permeation | 5.8 | 19.1 | 18.2 | 36.1 | 29.8 |
| after 3 hours (μg/cm²) | | | | | |
| Ratio of the cumulative | 1.0 | 3.3 | 3.1 | 6.2 | 5.1 |
| amount of permeation | | | | | |

### (Results)

According to the results in Table 4 and FIG. 2, it was found that the permeability of the particular water-soluble agent into the pseudo skin membrane, in other words, the transdermal permeability, can be remarkably increased in cases where an amino acid surfactant or a taurine surfactant is used.

1. A cosmetic comprising:
at least one water-soluble agent selected from the group consisting of an imidazolidinone derivative of the following Formula 1: 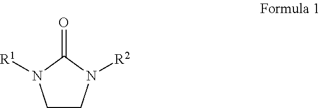
(wherein in Formula 1, R¹ and R² are each independently a hydrogen atom, a C₁ to C₄ linear or branched alkyl group having 0 to 3 hydroxyl groups, or a C₃ to C₇ cycloalkyl group having 0 to 5 hydroxyl groups)
and a salt thereof; and
at least one surfactant selected from the group consisting of an amino acid surfactant and a taurine surfactant.

2. The cosmetic according to claim 1, wherein the amino acid surfactant is an α-amino acid surfactant.

3. The cosmetic according to claim 1, wherein the surfactant is at least one selected from the group consisting of lauryl betaine, coconut oil fatty acid amidopropyl betaine, sodium cocoyl glycinate, potassium cocoyl glutamate, and coconut oil fatty acid sodium methyl taurate.

4. The cosmetic according to claim 1, wherein the imidazolidinone derivative is at least one selected from the group consisting of 2-imidazolidinone, 1-methyl-2-imidazolidinone, 1,3-dimethyl-2-imidazolidinone, 1-(2-hydroxyethyl)-2-imidazolidinone, and 1,3-bis-(2-hydroxyethyl)-2-imidazolidinone.

5. The cosmetic according to claim 1, wherein the mass ratio of the surfactant to the water-soluble agent is more than 0.05.

6. The cosmetic according to claim 1, wherein the content of the water-soluble agent in the total cosmetic is 0.1% by mass or more.

7. A beauty method comprising applying the cosmetic according to claim 1 to skin.

8. A method of enhancing transdermal permeation for at least one water-soluble agent selected from the group consisting of an imidazolidinone derivative of the following Formula 1: 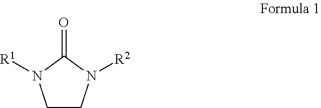
(wherein in Formula 1, R¹ and R² are each independently a hydrogen atom, a C₁ to C₄ linear or branched alkyl group having 0 to 3 hydroxyl groups, or a C₃ to C₇ cycloalkyl group having 0 to 5 hydroxyl groups)
and a salt thereof, comprising adding at least one surfactant selected from the group consisting of an amino acid surfactant and a taurine surfactant to a composition comprising the at least one water soluble agent of Formula 1.

9. The method according to claim 8, wherein the amino acid surfactant is an α-amino acid surfactant.

10. The method according to claim 8, wherein the surfactant is at least one selected from the group consisting of lauryl betaine, coconut oil fatty acid amidopropyl betaine, sodium cocoyl glycinate, potassium cocoyl glutamate, and coconut oil fatty acid sodium methyl taurate.

11. The method according to claim 8, wherein the imidazolidinone derivative is at least one selected from the group consisting of 2-imidazolidinone, 1-methyl-2-imidazolidinone, 1,3-dimethyl-2-imidazolidinone, 1-(2-hydroxyethyl)-2-imidazolidinone, and 1,3-bis-(2-hydroxyethyl)-2-imidazolidinone.
